(19) United States
(12) Patent Application Publication
(10) Pub. No.: US 2014/0323732 A1
(43) Pub. Date: Oct. 30, 2014

(54) NEW PROCESS FOR THE PRODUCTION OF TIOTROPIUM SALTS

(71) Applicants: Joerg BRANDENBURG, Wiesbaden (DE); Waldemar PFRENGLE, Biberach an der Riss (DE)

(72) Inventors: Joerg BRANDENBURG, Wiesbaden (DE); Waldemar PFRENGLE, Biberach an der Riss (DE)

(73) Assignee: BOEHRINGER INGELHEIM PHARMA GMBH & CO. KG, Ingelheim am Rheim (DE)

(21) Appl. No.: 14/324,610

(22) Filed: Jul. 7, 2014

Related U.S. Application Data
(63) Continuation of application No. 13/727,799, filed on Dec. 27, 2012, now abandoned.
Application No. 13/727,799 is a continuation of application No. 12/769,927, filed on Apr. 29, 2010, now abandoned.
Application No. 12/769,927 is a continuation of application No. 11/459,457, filed on Jul. 24, 2006, now abandoned.

(30) Foreign Application Priority Data
Jul. 27, 2005 (DE) 10 2005 035 112

(51) Int. Cl. C07D 451/02

(52) U.S. Cl. CPC C07D 451/02
USPC 546/91

(57) ABSTRACT

The invention relates to a new process for preparing tiotropium salts of general formula 1

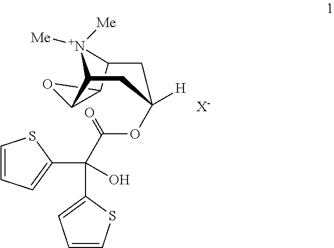

wherein X⁻ may have the meanings given in the claims and in the specification.

The invention relates to a new process for preparing tiotropium salts of general formula 1

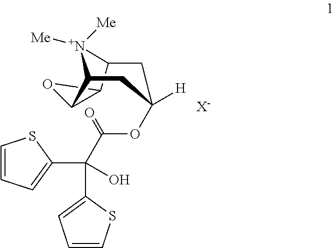

wherein X⁻ may have the meanings given in the claims and in the specification.

### BACKGROUND OF THE INVENTION

Anticholinergics may be used to advantage to treat a number of diseases. Particular to mention may be made for example of the treatment of asthma or COPD (chronic obstructive pulmonary disease). Anticholinergics which have a scopine, tropenol or tropine basic structure are proposed for example by WO 02/03289 for the treatment of these diseases. Moreover, tiotropium bromide is particularly disclosed in the prior art as a highly potent anticholinergic. Tiotropium bromide is known for example from EP 418 716 A1.

In addition to the methods of synthesis for preparing scopine esters, disclosed in the prior art mentioned above, a process for preparing esters of scopine is disclosed particularly in WO03/057694.

The aim of the present invention is to provide an improved industrial method of synthesis which enables the compounds of general formula 1 to be synthesised more easily, in a manner which is an improvement on the prior art.

### DETAILED DESCRIPTION OF THE INVENTION

The present invention relates to a process for preparing tiotropium salts of formula 1

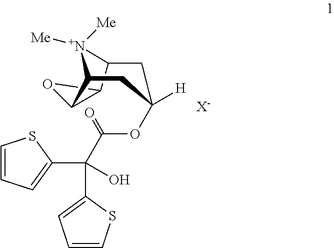

wherein

X⁻ may represent an anion with a single negative charge, preferably an anion selected from among the chloride, bromide, iodide, sulphate, phosphate, methanesulphonate, nitrate, maleate, acetate, citrate, fumarate, tartrate, oxalate, succinate, benzoate, p-toluenesulphonate and trifluoromethanesulphonate,

characterised in that a compound of formula 2

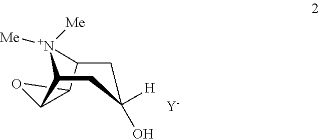

wherein

Y⁻ denotes a lipophilic anion with a single negative charge, preferably an anion selected from among the hexafluorophosphate, tetrafluoroborate, tetraphenylborate and saccharinate, particularly preferably hexafluorophosphate or tetraphenylborate

is reacted in one step with a compound of formula 3

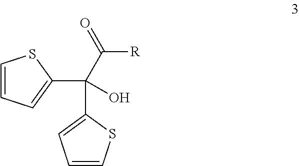

wherein

R denotes a group selected from among methoxy, ethoxy, propoxy, isopropoxy, isopropenyloxy, butoxy, O—N-succinimide, O—N-phthalimide, phenyloxy, nitrophenyloxy, fluorophenyloxy, pentafluorophenyloxy, vinyloxy, 2-allyloxy, —S-methyl, —S-ethyl and —S-phenyl,

in a suitable solvent with the addition of a suitable base to form a compound of formula 4

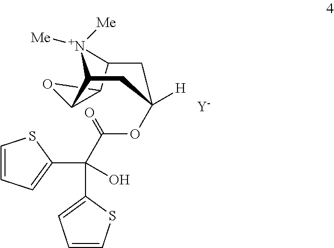

wherein the group Y⁻ may have the meanings given above, and without isolation the compound of formula 4 is converted into the compound of formula 1 by reaction with a salt cat⁺X⁻, wherein cat⁺ denotes a cation selected from among the Li⁺, Na⁺, K⁺, Mg²⁺, Ca²⁺, organic cations with quaternary N (e.g. N,N-dialkylimidazolium, tetraalkylammonium) and X⁻ may have the meanings given above.

Preferably the present invention relates to a process for preparing tiotropium salts of formula 1 , wherein

X⁻ may represent an anion with a single negative charge selected from among the chloride, bromide, iodide, methanesulphonate, p-toluenesulphonate and trifluoromethanesulphonate, preferably chloride, bromide, iodide, methanesulphonate or trifluoromethanesulphonate, particularly preferably chloride, bromide or methanesulphonate, particularly preferably bromide.

A particularly preferred process according to the invention is characterised in that the reaction is carried out with a compound of formula 3, wherein

R denotes a group selected from among methoxy, ethoxy, propoxy, isopropoxy, isopropenyloxy, butoxy, O—N-succinimide, O—N-phthalimide, phenyloxy, nitrophenyloxy, fluorophenyloxy, pentafluorophenyloxy, vinyloxy and 2-allyloxy.

A particularly preferred process according to the invention is characterised in that the reaction is carried out with a compound of formula 3, wherein

R denotes a group selected from among methoxy, ethoxy, propoxy, isopropoxy, isopropenyloxy, butoxy, O—N-succinimide, O—N-phthalimide, vinyloxy and 2-allyloxy, preferably selected from methoxy, ethoxy, propoxy, and butoxy, particularly preferably methoxy or ethoxy.

A particularly preferred process according to the invention is characterised in that the reaction is carried out with a compound of formula 2, wherein

Y⁻ may represent an anion with a single negative charge selected from among the hexafluorophosphate, tetrafluoroborate and tetraphenylborate, preferably hexafluorophosphate.

A particularly preferred process according to the invention is characterised in that the final reaction of the compound of formula 4 to obtain the compound of formula 1 is carried out with the aid of a salt catX, wherein cat⁺ is selected from among Li⁺, Na⁺, K⁺, Mg²⁺, Ca²⁺, organic cations with quaternary N (e.g. N,N-dialkylimidazolium, tetraalkylammonium) and wherein X⁻ may have the meanings given above.

The term alkyl groups, including those which are part of other groups, refers to branched and unbranched alkyl groups with 1 to 4 carbon atoms. Examples include: methyl, ethyl, propyl, butyl. Unless otherwise stated, the terms propyl and butyl used above include all the possible isomeric forms thereof. For example the term propyl includes the two isomeric groups n-propyl and iso-propyl, while the term butyl includes n-butyl, iso-butyl, sec. butyl and tert.-butyl.

The term alkoxy or alkyloxy groups refers to branched and unbranched alkyl groups with 1 to 4 carbon atoms which are linked by an oxygen atom. Examples include: methoxy, ethoxy, propoxy, butoxy. Unless otherwise stated, the above-mentioned terms include all the possible isomeric forms.

The terms phenyl-methyl and phenyl-NO₂ denote phenyl rings which are substituted by methyl or NO₂. All the possible isomers are included (ortho, meta or para), while para- or meta-substitution are of particular interest.

The term cycloalkyl groups refers to cycloalkyl groups with 3-6 carbon atoms, for example cyclopropyl, cyclobutyl, cyclopentyl or cyclohexyl.

The term lipophilic anions according to the invention in this case refers to anions of the kind whose sodium or potassium salts have a solubility in polar organic solvents such as methanol or acetone of >1 wt.-%.

The process according to the invention is particularly characterised in that it can be carried out in relatively non-polar solvents, by virtue of the solubility of the starting compounds of formula 2 and the intermediates of formula 4. This allows the reaction to be carried out under very gentle conditions, with fewer side reactions compared with reactions carried out in highly polar aprotic solvents with the delicate tiotropium salts and consequently a higher yield.

The reaction of the compounds of formula 2 with the compounds of formula 3 is preferably carried out in an aprotic organic solvent, preferably in a slightly polar organic solvent. Particularly preferred solvents which may be used according to the invention are acetone, pyridine, acetonitrile and methylethylketone, of which acetone, acetonitrile and pyridine are preferably used. Particularly preferably the reaction is carried out in a solvent selected from among acetone and acetonitrile, while the use of acetone is particularly preferred according to the invention.

It may optionally be advantageous to activate the reaction of the compound of formula 2 with 3 by the addition of a catalyst. Particularly gentle activation is made possible according to the invention by the use of catalysts selected from among the zeolites, lipases, tert. amines, such as for example N,N-dialkylamino-pyridine, 1,4-diazabicyclo[2,2,2]octane (DABCO) and diisopropylethylamine and alkoxides, such as, for example, [sic] while the use of zeolites and particularly zeolites and potassium-tert.-butoxide is particularly preferred according to the invention. Particularly preferred zeolites are molecular sieves selected from among the molecular sieves of a basic nature consisting of sodium-or potassium-containing aluminosilicates, preferably molecular sieves of the empirical formula _{Na}₁₂[(AlO₂)₁₂(SiO₂)₁₂]xH₂O, while the use of molecular sieve type 4A (indicating a pore size of 4 Angström) is particularly preferred according to the invention.

The reaction of 2 with 3 to obtain the compound of formula 4 may be carried out at elevated temperature depending on the type of catalyst. Preferably the reaction is carried out at a temperature of 30° C., particularly preferably in the range from 0 to 30° C.

The compounds of formula 3 may be obtained by methods known from the prior art. Mention may be made for example of WO03/057694, which is hereby incorporated by reference.

The compounds of formula 2 are of central importance to the process according to the invention. Accordingly, in another aspect the present invention relates to compounds of formula 2

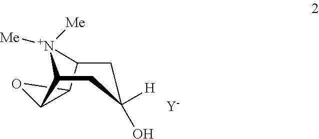

as such, wherein

Y⁻ denotes a lipophilic anion with a single negative charge, preferably an anion selected from among the hexafluorophosphates, tetrafluoroborate, tetraphenylborate and saccharinate, particularly preferably hexafluorophosphates or tetraphenylborate

The following method may be used to prepare the compounds of formula 2. Preferably a scopine salt of formula 5,

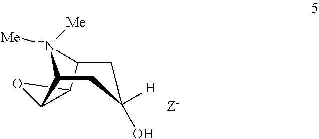

wherein Z⁻ denotes an anion with a single negative charge which is different from Y⁻, is dissolved in a suitable solvent, preferably in a polar solvent, particularly preferably in a solvent selected from among the water, methanol, ethanol, propanol or isopropanol. According to the invention water and methanol are preferred as the solvent, while water is of exceptional importance according to the invention.

Particularly preferred starting compounds for preparing the compound of formula 2 are those compounds of formula 5, wherein

Z⁻ denotes an anion with a single negative charge, preferably an anion selected from among the chloride, bromide, iodide, sulphate, phosphate, methanesulphonate, nitrate, maleate, acetate, citrate, fumarate, tartrate, oxalate, succinate, benzoate and p-toluenesulphonate.

Also preferred as starting compounds for preparing the compound of formula 2 are those compounds of formula 5, wherein

Z⁻ may represent an anion with a single negative charge selected from among chloride, bromide, 4-toluenesulphonate and methanesulphonate, preferably bromide.

The solution thus obtained is mixed with a salt cat′Y. Y here denotes one of the above-mentioned anions wherein cat′ denotes a cation which is preferably selected from among protons (H⁺), alkali or alkaline earth metal cations, ammonium, preferably protons or alkali metal cations, particularly preferably Li⁺, Na⁺- and K⁺ ions.

Preferably according to the invention 1 mol, preferably 1-1.5 mol, optionally 2-5 mol of the salt cat′Y is used per mol of the compound of formula 5 used. It is clear to the skilled man that it is possible to use smaller amounts of the salt cat′Y, but that this may then lead to only partial reaction of the compound of formula 5.

The resulting solution is stirred until the reaction is complete. The work may be done at ambient temperature (about 23° C.) or optionally also at slightly elevated temperature in the range from 25-50° C. After the addition is complete, and to some extent during the addition as well, the compounds of formula 2 crystallise out of the solution. The products obtained may, if necessary, be purified by recrystallisation from one of the above-mentioned solvents. The crystals obtained are isolated and dried in vacuo.

In another aspect the present invention relates to the use of compounds of formula 2 as starting compounds for preparing compounds of formula 1. In another aspect the present invention relates to the use of compounds of formula 2 as starting compounds for preparing compounds of formula 4. In another aspect the present invention relates to the use of compounds of formula 5 as starting compounds for preparing compounds of formula 2. In another aspect the present invention relates to the use of compounds of formula 5 as starting compounds for preparing compounds of formula 4.

In another aspect the present invention relates to a process for preparing compounds of formula 1, characterised in that a compound of formula 2 is used as a starting compound for preparing compounds of formula 1. In another aspect the present invention relates to a process for preparing compounds of formula 4, characterised in that a compound of formula 2 is used as a starting compound for preparing compounds of formula 4.

In another aspect the present invention relates to a process for preparing compounds of formula 2, characterised in that a compound of formula 5 is used as a starting compound for preparing compounds of formula 2.

In another aspect the present invention relates to a process for preparing compounds of formula 4, characterised in that a compound of formula 5 is used as a starting compound for preparing compounds of formula 4.

The compounds of formula 4 are of central importance to the process according to the invention. Accordingly, in another aspect, the present invention relates to compounds of formula 4

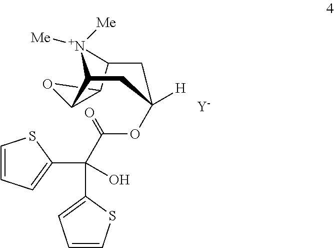

per se, wherein the group Y⁻ may have the meanings given above.

In another aspect the present invention relates to the use of compounds of formula 4 as starting compounds for preparing compounds of formula 1. In another aspect the present invention relates to a process for preparing compounds of formula 1, characterised in that a compound of formula 4 is used as a starting compound for preparing compounds of formula 1.

The compounds of formula 4 are obtained as hereinbefore described within the scope of the process according to the invention for preparing compounds of formula 1 as intermediates. Within the scope of the process according to the invention for preparing to compounds of formula 1 , in a preferred embodiment of the invention, the compound of formula 4 does not have to be isolated.

### EXAMPLES

The Examples that follow serve to illustrate some methods of synthesis carried out by way of example. They are to be construed only as possible methods described by way of example without restricting the invention to their contents.

### Example 1

### N-methylscopinium Hexafluorophosphate

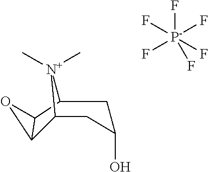

N-methylscopinium bromide is dissolved in water and combined with an equimolar or molar excess of a water-soluble hexafluorophosphate (sodium or potassium salt). (Aqueous hexafluorophosphoric acid also leads to precipitation).

The N-methylscopinium hexafluorophosphate is precipitated/crystallised as a white, water-insoluble product, it is isolated, optionally washed with methanol and then dried at about 40° C. in the drying cupboard.

M.p.: 265-267° C. (melting with discoloration);

H-NMR: in acetonitrile-d3 σ(ppm): 1.9 (dd, 2H) , 2.55(dd, 2H), 2.9 (s,3H), 3.29 (s,3H), 3.95(dd, 4H), 3.85 (s, 1H).

### Example 2

### Tiotropium Bromide

1.6 g (5mmol) methylscopinium hexafluorophosphate (Example 1) and 2.0 g (7.8 mmol) methyl dithienylglycolate are refluxed in 50 ml acetone and in the presence of 10 g molecular sieve 4A for 50-70 hours.

The reaction mixture is filtered, the filtrate is combined with a solution of 0.3 g of LiBr in 10 ml acetone. The still unreacted N-methylscopinium bromide that crystallises out is separated off by filtration. After the addition of another 0.6 g LiBr (dissolved in acetone) tiotropium bromide is precipitated in an isolated yield of 30% (based on the compound of Example 1 used).

### Example 3

### Tiotropium Hexafluorophosphate

Tiotropium hexafluorophosphate is not isolated within the scope of the reaction according to Example 2 but further reacted directly to obtain the tiotropium bromide.

For the purposes of characterising tiotropium hexafluorophosphate this compound was specifically prepared and isolated. The following characteristic data were obtained.

M.p.: 233-236° C. (melting with discoloration)

H-NMR: in acetone-d6: σ(ppm): 2.08 (dd, 2H) , 2.23(dd, 2H), 3.32 (s, 3H), 3.50 (s, 3H), 3.62(s,2H), 4.28(m, 2H), 5.39(m, 1H) 0.6.25 (s), 7.02(m,2H), 7.027.22(m,2H), 7.46(m,2H), P-NMR: in acetone-d6: σ(ppm): −143.04, heptet, J=4.37.

### Example 4

### Tiotropium Bromide

31.5 g (100 mmol) methylscopinium hexafluorophosphate (Example 1) and 25.4 g (100 mmol) methyl dithienylglycolate are refluxed in 400 ml acetone and in the presence of 40 g of powdered molecular sieve 4A (Fluka) and DMAP (4,4-dimethylaminopyridine) for 24 h. (The molecular sieve was replaced after 3 h by an equal amount.) The reaction mixture is filtered, washed with 200 ml acetone, the filtrate is combined stepwise with a solution of 9.6 g LiBr (110 mmol) in 110 ml acetone. The still unreacted N-methylscopinium bromide that crystallises out is separated off by filtration (fractionated precipitation). The crystal fractions were filtered off and dried. The composition of the fractions was determined by thin layer chromatography. Tiotropium bromide in an isolated yield of 16.6 g (35%) (based on the compound according to Example 1 used). Purity HPLC>99%. Purity according to TLC: no detectable contamination.

### Example 5

### Tiotropium Bromide

1.6 g (5 mmol) methylscopinium hexafluorophosphate (Example 1) and 1.25 g (5 mmol) methyl dithienylglycolate are stirred in 50 ml acetone and in the presence of 2 g powdered molecular sieve 4A (Fluka) and 6 mg potassium-tert.-butoxide at 0° C. for 4 h.

The reaction mixture is filtered, washed with 20 ml acetone, the filtrate is combined stepwise with a solution of 0.7 g LiBr (13 mmol) in 11 ml acetone. The unreacted material that crystallises out is separated off by filtration (fractionated precipitation). The crystal fractions were filtered off and dried. The composition of the fractions was determined by thin layer chromatography. The tiotropium bromide fractions were suction filtered, washed with acetone, recrystallised from water, washed with acetone and dried. 1.2 g (48% yield based on the compound according to Example 1 used). Tiotropium bromide was isolated in this way.

Purity HPLC: 99.8%, TLC: no visible contamination

### Example 6

### Tiotropium Bromide

31.5 g (0.1 mol) methylscopinium hexafluorophosphate (Example 1) and 30.5 g (0.10 mol) 2,2′- methyl dithienylglycolate are dissolved in 400 ml acetone and stirred in the presence of 90 g of zeolite of type 4A (Na₁₂Al₁₂Si₁₂O₄₈xnH₂O) and 0.2 g (1 mmol) potassium-tert.-butoxide over a period of 20-24 hours at 0° C.

The reaction mixture is filtered, the filtrate is combined with a solution of 8.7 g LiBr (8.7 g 0.10 mol in 100 ml acetone).

The product that crystallises out is separated off by filtration, washed with acetone and then dried.

41.4 g (87.7%) yield is obtained, with a conversion level of 90%.

### Example 7

### N-methylscopinium Tetraphenylborate

20 g (80 mmol) methylscopinium bromide are dissolved in 500 ml of methanol. 27.38 (80 mmol) sodium tetraphenylborate, dissolved in 150 ml of methanol, are metered in. The suspension obtained is stirred for 10 min at ambient temperature and filtered. The crystals separated off are washed with 50 ml of methanol and dried.

Yield: 39.1 g (91.73% yield); M.p.: 261° C.

### Example 8

### Tiotropium Tetraphenylborate

0.245 g (0.5 mmol) methylscopinium tetraphenylborate (Example 7), and 0.154 g (0.6 mmol) 2,2-methyl dithienylglycolate are dissolved in 25 ml acetone and stirred in the presence of 1.0 g zeolite of type 4A (Na₁₂Al₁₂Si₁₂O₄₈ xnH₂O) and 5 mg of potassium tert.-butoxide over a period of 20-30 hours at 0° C.

According to HPLC 79% of the 2,2-methyl dithienylglycolate reacted are converted after 26 h into tiotropium tetraphenylborate. (Non-isolated yield: 43%).

The reactions mentioned by way of example take place with virtually no formation of by-products. If it is desired that the reactions should take place without total reaction of the starting materials, the N-methylscopinium bromide isolated in the first step of working up may therefore be recycled into the reaction according to Example 1, thereby significantly increasing the total yield within the scope of a production process.

1. A process for preparing tiotropium salts of formula 1 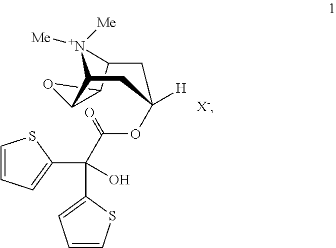
wherein X⁻ represents bromide, comprising:
reacting in one step a compound of formula 2 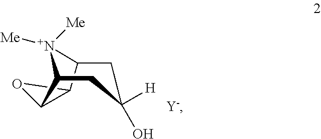
wherein Y⁻ is hexafluorophosphate,
with a compound of formula 3 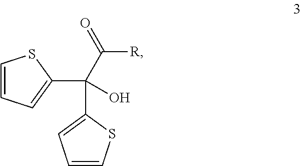
wherein R is methoxy or ethoxy,
in a solvent selected from acetone, acetonitrile or pyridine with the addition of a catalyst selected from zeolites to obtain a compound of formula 4 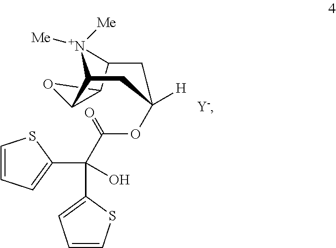
wherein the group Y⁻ has the meaning given above, and
without being isolated, the compound of formula 4 is converted into the compound of formula 1 by reacting formula 4 with a salt cat³⁰ X⁻, wherein cat⁺ denotes a cation selected from the group consisting of Li⁺, Na⁺, and K⁺, and X⁻ has the meaning given above.

2-11. (canceled)

12. The process according to claim 1, wherein formula 2 is used as a starting compound for preparing compounds of formula 1.

13. (canceled)

14. The process according to claim 1, wherein formula 4 is used as a starting compound for preparing compounds of formula 1.
